(19) United States
(12) Patent Application Publication
(10) Pub. No.: US 2022/0378616 A1
(43) Pub. Date: Dec. 1, 2022

(54) Corneal Implant

(71) Applicant: Chandrashekar Balachandran, Killara (AU)

(72) Inventor: Chandrashekar Balachandran, Killara (AU)

(21) Appl. No.: 17/741,341

(22) Filed: May 10, 2022

Related U.S. Application Data
(63) Continuation-in-part of application No. 16/118,843, filed on Aug. 31, 2018, now Pat. No. 11,324,587.

(30) Foreign Application Priority Data
Aug. 31, 2017 (AU) 2017903503

(51) Int. Cl. A61F 9/013; A61F 2/14

(52) U.S. Cl. CPC A61F 9/0133; A61F 2/142

(57) ABSTRACT

A method for performing a corneal transplantation comprising the steps of: inserting at least part of a corneal implant into the anterior chamber of the eye, the corneal implant comprising an implant portion and a manipulating portion, the manipulating portion comprising non-endothelial tissue; positioning the implant portion of the implant to adhere to the posterior surface of the cornea using the manipulating portion.

### CROSS-REFERENCE TO RELATED APPLICATIONS

This application is a continuation-in-part of U.S. Ser. No. 16/118,843 for “Corneal Implant” filed Aug. 31, 2018, which in turn claims priority from Australian Application No. 2017903503 filed Aug. 31, 2017, entitled “Corneal Implant,” each of which is hereby incorporated herein by reference in its entirety.

### TECHNICAL FIELD

The present invention relates to a corneal implant and a method and apparatus for corneal transplantation.

### BACKGROUND SECTION

The cornea is the front window of the eye. It consists of multiple layers. From superficial to deep they are the epithelium, stroma and endothelium. Each layer can be affected by different diseases. The corneal endothelium can be affected in diseases such as Fuchs' endothelial dystrophy and pseudophakic bullous keratopathy which result in the reduction on corneal endothelial cell density. The corneal endothelium consists of a monolayer of cells that do not replicate. Therefore, they can only be replaced by transplantation of cells from another person's cornea.

Transplantation techniques are evolving. They range from full thickness grafts (i.e. penetrating keratoplasty) to partial thickness grafts such as Descemet Stripping Endothelial Keraplasty (DSEK/DSAEK) to just the monolayer of corneal endothelium-Descemet's membrane complex called Descement Membrane Endotheial Keratoplasy (DMEK). The endothelium-descement graft complex is 10-15 microns thick. By replacing only that which is needed, smaller incisions can be made and less astigmatism can be induced such as with penetrating keratoplasty. The graft host interface is also kept clear of haze reducing risk of visual disturbance which may be seen with DSEK/DSAEK.

DMEK surgery involves a few challenges. Firstly, the graft needs to be harvested without touching the endothelium with instruments. Once harvested, the endothelium-descemet complex immediately rolls into either a single or a double scroll with the endothelium facing outwards. This scroll has to be inserted in the anterior chamber, centered and unrolled with the endothelium facing posteriorly. All these maneuvers have to be conducted without any instruments touching the graft. Instead, balanced salt solution &/or air injected through a cannula is used to manipulate the Descemet endothelial graft complex. Once unrolled and centered, air is injected deep to the graft to raise the graft to the posterior surface of the cornea. Air is left in the anterior chamber for an hour for the graft to adhere.

### SUMMARY OF THE INVENTION

In a first aspect the invention provides a corneal implant comprising a portion of corneal endothelial tissue and a portion of scleral tissue.

Preferably the portion of endothelial tissue is disk shaped.

Preferably the portion of scleral tissue extends from the edge of the portion of endothelial tissue.

Preferably the portion of scleral tissue is tail shaped.

Preferably the corneal implant comprises a tail portion, the tail portion being sceleral tissue.

Preferably the portion of corneal endothelial tissue and the portion of scleral tissue are integral.

Preferably the corneal implant is keyhole shaped.

Preferably the corneal implant comprises a perforated section.

Preferably the perforated section is within the portion of scleral tissue.

Preferably the perforated section is positioned at a junction of the portions of the corneal endothelial tissue and the sclera tissue.

Preferably the thickness of the portion of corneal endothelial tissue is in the range of 10-15 μm.

Preferably the corneal implant is cut from a cornea.

Preferably the corneal implant is suitable for insertion into the anterior chamber of an eye and for adhering to the posterior surface of the cornea.

Preferably the implant comprises a disc shape and having a tail extending from the disk.

Preferably the portion of scleral tissue is comprised within the tail.

Preferably the portion of corneal endothelial tissue comprises corneal endothelium and Descement membrane.

In a second aspect the invention provides a method for preparation of a corneal implant comprising the steps of harvesting a corneal implant from the posterior surface of a cornea, the corneal implant comprising a portion of corneal endothelial tissue and a portion of scleral tissue.

Preferably the step of harvesting comprises the steps of applying a trephination to the posterior surface of the cornea to harvest the corneal implant.

Preferably the trephination is decentered on the posterior surface of the cornea.

In a third aspect the invention provides a corneal implant comprising an implant portion and a manipulating portion.

Preferably the implant portion comprises corneal endothelial tissue.

Preferably the manipulating portion comprises scleral tissue.

Preferably the corneal implant comprises a disc portion and a tail portion, wherein the tail portion comprises the manipulating portion.

In a fourth aspect the invention provides a method for performing a corneal transplantation comprising the steps of: a. inserting at least part of a corneal implant into the anterior chamber of the eye, the corneal implant comprising an implant portion and a manipulating portion, the manipulating portion comprising non-endothelial tissue; b. positioning the implant portion of the implant to adhere to the posterior surface of the cornea using the manipulating portion.

Preferably the implant portion comprises corneal endothelial tissue.

Preferably the implant portion is disc shaped.

Preferably the manipulating portion extends from the edge of the implant portion.

Preferably the manipulating portion is tail shaped.

Preferably the manipulating portion comprises scleral tissue.

Preferably the step of inserting comprises positioning the implant portion into the anterior chamber of the eye and retaining at least part of the manipulating portion outside of the eye.

Preferably the implant portion is scroll shaped during insertion.

Preferably the step of insertion comprises: a. using a syringe to inject the corneal implant into the anterior chamber of the eye.

Preferably the corneal implant is inserted into the anterior chamber of the eye through an incision in the sclera.

Preferably a suture is placed in the manipulating portion.

Preferably the step of placing a suture in the manipulating portion closes the incision.

In a fifth aspect the invention provides a method for performing a corneal transplantation comprising the steps of: a. inserting at least part of a corneal implant according to the first aspect and the third aspect into the anterior chamber of the eye; b. positioning the implant portion of the implant to adhere to the posterior surface of the cornea.

In a sixth aspect the invention provides a kit comprising the corneal implant of any one the first aspect and the third aspect and a fluid.

Embodiments further comprise a delivery device for inserting the corneal implant into the anterior portion of an eye.

In a further embodiment the invention provides a corneal trephine comprising a tubular body and a cross section having a rounded portion and an elongated tail portion extending from the rounded portion, the tubular body having a leading edge configured to cut into a cornea.

The cross section may have a major axis and a minor axis, the major axis encompassing the tail portion and the rounded portion.

The cross section may be symmetrical about the major axis.

The leading edge may have a convex curvature.

The convex curvature is configured to match the curvature of a cornea.

The convex curvature of the leading edge extends from low points on the leading edge at or around the points at which the major axis intersects the leading edge to crest points on the leading edge at or towards the mid point of the major axis.

The complex curvature is symmetrical about the major axis.

The leading edge is a cutting blade.

In a further aspect the invention provides a kit comprising a corneal trephine and a punch block, the punch block having a curved surface, wherein the curvature of the leading edge is complimentary to the curved surface of the punch block. The kit further comprising an alignment device to align the trephine with respect to the punch block.

### BRIEF DESCRIPTION OF THE DRAWINGS

The foregoing features of embodiments will be more readily understood by reference to the following detailed description, taken with reference to the accompanying drawings, in which:

FIG. 1 is an illustration of the basic structure of a human eye;

FIG. 1a is an exploded section of the cornea;

FIG. 2 shows the corneal implant within a donor cornea;

FIG. 3 illustrates a corneal implant;

FIG. 4 is a side view of a trephine;

FIG. 5 is a top angle view of the cutting surface of the trephine;

FIG. 6 is a cross-sectional view of the trephine including dimensions;

FIG. 7 shows a corneal implant;

FIG. 8 shows insertion of the corneal implant into an anterior chamber of the eye; and

FIG. 9 shows a corneal implant positioned within the eye.

FIG. 10 shows an embodiment of a trephine.

### DETAILED DESCRIPTION OF THE DRAWINGS

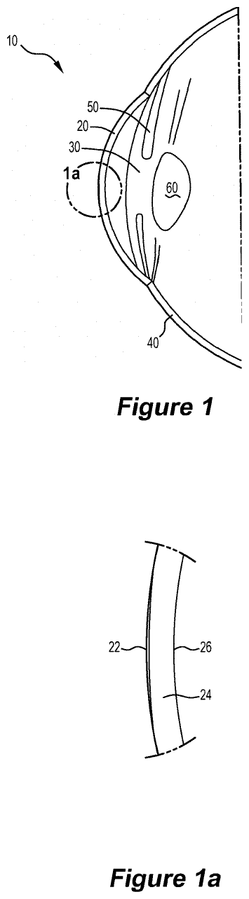

FIG. 1 shows the basic structure of the eye. FIG. 1 is a cross-sectional representation of the front section of the eye. The cornea 20 is the transparent front part of the eye. The cornea is a circular section and surrounded on all sides by sclera 40. The sclera is the white of the eye. Behind the cornea is the anterior chamber 30. The iris 50 and lens 60 are positioned behind cornea 20.

The cornea has multiple layers as shown in exploded view in FIG. 1a. The outer layer is the corneal epithelium 22. The thick transparent middle layer is the corneal stroma 24. The rear layer of the cornea is the corneal endothelium. The corneal endothelium is a single layer of cells having a typical thickness of between 5 to 15 microns. The cells of the corneal endothelium do not replicate. The corneal endothelium can be affected in diseases such as Fuchs' endothelial dystrophy which can result in the reduction of corneal endothelial cell density. Deterioration of the corneal endothelium can cause a gradual decline in vision and clouding.

We now describe a new treatment to replace corneal endothelium tissue using a corneal implant.

In the present invention replacement of corneal endothelium is performed by creating a corneal implant from a donor eye.

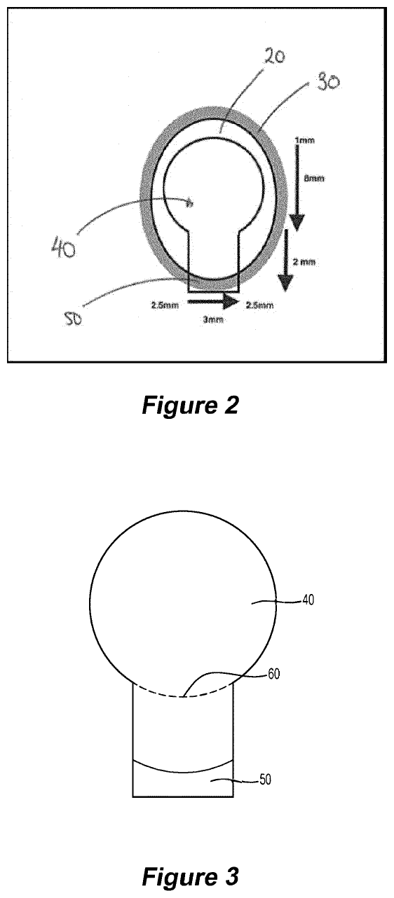

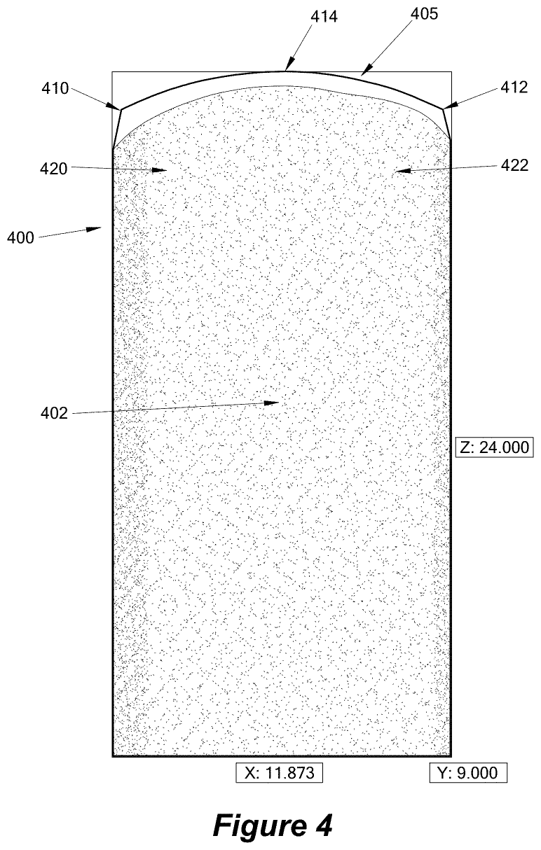

FIG. 2 shows a cornea 20 and surrounding portion of sclera 30. Typically this portion of the eye is removed from the donor eye and positioned with the endothelial tissue facing upward in trephine punch block. A keyhole shaped trephine (shown in FIGS. 4-7) is applied to the posterior side of the cornea covered with the layer of corneal endothelium to cut the corneal implant from the donor cornea. The resulting corneal implant after extraction from the cornea is shown in FIG. 3.

As illustrated in FIG. 2, the corneal implant includes a portion of corneal endothelial tissue 40 and a portion of sclera tissue 50. As shown in FIG. 2 the trephine is decentered with respect to the donor cornea in order to harvest a central disc with as long tail as possible.

As illustrated in FIGS. 2 and 3 the corneal implant is generally keyhole shaped. The keyhole includes a disc shaped portion 40. The implant includes a tail shaped extension from the edge of the disc shaped portion 50.

In an embodiment of FIGS. 2 and 3 the implant portion comprising sclera tissue 50 is only a small portion at the end of the tail section. In further embodiments the trephine may be arranged to allow a different percentage portion of the tail section to be cut from the sclera part of the eye and so the junction between the endothelia tissue and sclera tissue may be at a different position within the tail. In a further embodiment the tail may not include any scleral tissue. The implant is cut in a single portion and the disc and tail portions of the implant are integral.

Example dimensions for the implant are shown in FIG. 2. These dimensions are for illustrative purposes only and are not limiting. In particular the dimensions may be determined specifically for patient undergoing surgery depending on the size of the eye for surgery, the size of the donor eye and the size of the endothelial tissue requiring transplant. In the example of FIG. 2 the disc shaped portion of the implant has a diameter of around 8 mm. The tail portion has a length of around 2 mm and width of around 3 mm.

In a further embodiment the above non circular corneal implant shape could be generated using a laser to achieve the same design.

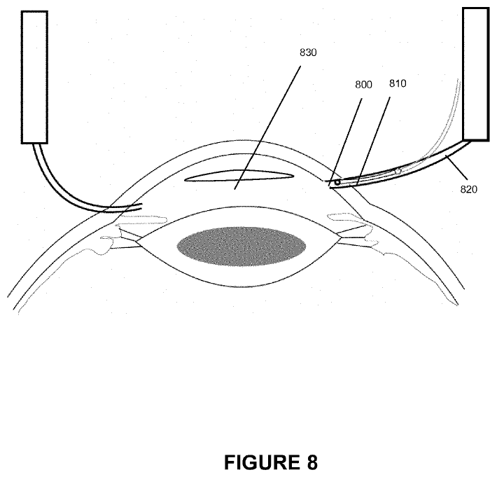

In some embodiments of the invention a perforated section 60 is incorporated into the implant. In the embodiment of FIG. 3, perforated section 60 is positioned at the junction between the disc shaped section of the corneal implant and the tail section. In further embodiments the perforated section may be positioned within the tail section of the implant. The perforated section is included in order that part of the tail section may be easily removed from the implant during surgery. This is discussed in more detail below with reference to FIGS. 7 and 8. In embodiments, the perforated section may be created using a needle or a laser. Typically, the perforated section includes a series of cuts through the implant in order to weaken the implant at that position. The perforations may be added after transplantation in to the use a small needle or a laser. The perforation is placed to remove the tail from the graft where necessary to do so. In the preferred embodiment, the perforation would be circumferential in position to allow the tail to be removed. In other embodiments a variable amount of the tail as preferred by the surgeon could be removed.

The preparation process of the implant involves a step of separating the endothelial layer from the stroma layer within the donor eye. This is performed by pealing the endothelial layer from the stroma layer. The graft is placed in a curved plastic well with vacuum generated using a syringe to hold the graft. Once secured the endothelium is separated by creating an incision close to the trabecular meshwork. It is then dissected forwards with blunt dissention breaking collagen attachments towards the center and separated from the underlying stroma. In one embodiment the endothelium is separated over the entire donor surface and then trephined. In another embodiment the endothelium is kept attached along a variable circumference of the sclera such as 2 mm so that the tail incorporates a section the sclera.

Further embodiments do not include perforations. In such embodiments the tail portion may be cut or torn away after transplantation.

In preferred embodiments all layers of the cornea are maintained during extraction of the corneal implant during trephination in order to maintain the integrity of the implant after the implant has been cut from the cornea the endothelia layer is peeled from the stroma layer. Once trephined the stroma is cut away from the descement endothelium complex near the tail. However, in further embodiments the endothelia layer may be separated from the stroma layer before the implant is cut from the donor eye. The corneal implant is then stained with methylene blue to help visualize the graft once it is inserted into the recipient eye.

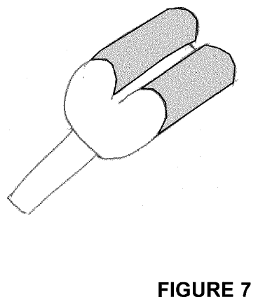

FIGS. 4, 5 and 6 show example illustrations of the keyhole shaped trephine used to cut the corneal implant from the donor cornea. The cutting procedure is performed in a sterile manner. Example dimensions are shown in FIG. 6. The keyhole implant shape is best illustrated in FIG. 6 showing a cross-sectional view through the trephine. The internal diameter of the disc section is 8 mm and the tail section has a width of 2 mm. The overall outer dimension for the disc region of the trephine is 9 mm and overall dimension including the disc and tail is 11.873 mm. The trephine has a cross-bar portion to maintain configuration of the trephine along its length. The cross-bar portion does not extend to the outermost portion of the trephine in order not to interfere with the corneal implant during cutting.

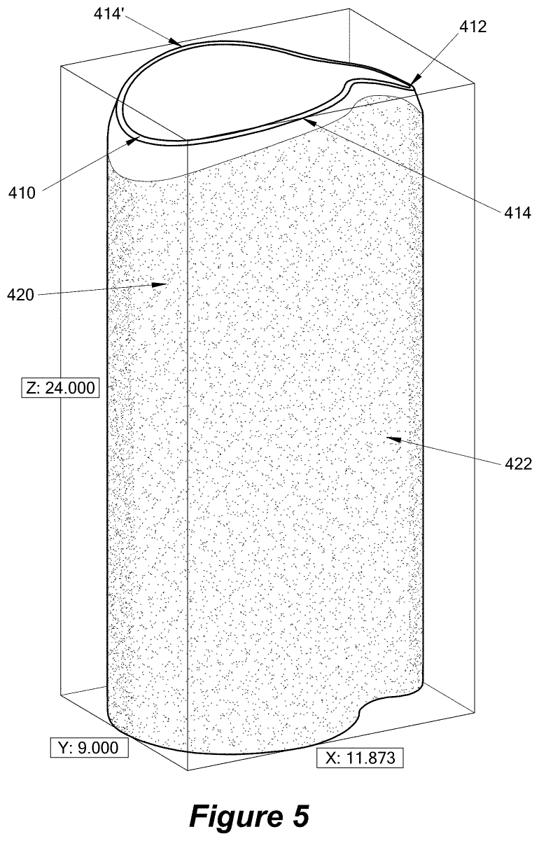

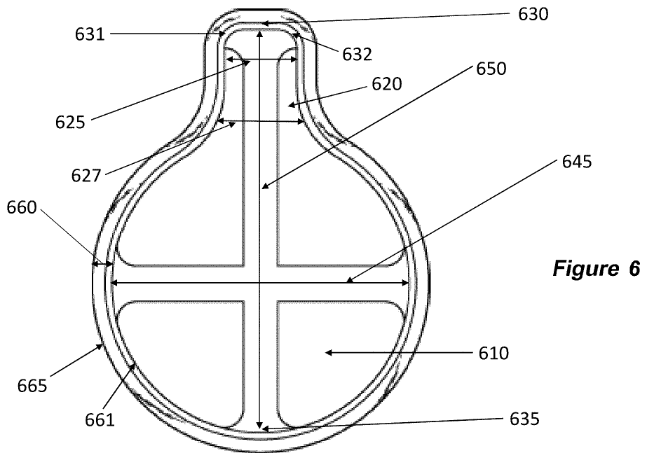

FIG. 5 shows a top perspective view across the cutting region of the trephine. As illustrated in FIG. 5 the cutting portion is rounded. The cutting surface is rounded in order to match the curvature of the posterior surface of the cornea. The curvature of the cutting edge would be approximately 7 mm to match the curvature of a normal cornea. This would be curvature of the punch block and would be mirrored in the trephine.

In embodiments the trephine is formed from cobalt chrome, which is used in hip prosthesis. Alternatively, another biocompatible metal would be used to make the trephine. The trephine is not inserted inside the patient.

The trephine is configured to cut a corneal implant from a donor cornea. The trephine may be referred to as a corneal trephine. The trephine may be referred to as a tubular trephine. The trephine may be referred to as a punch. The trephine may be referred to as a corneal punch.

The trephine 400 is tubular and has an elongated body portion 402. The trephine has a leading edge 405. The leading edge is the perimeter defining the end of the trephine. In the example of FIG. 4, the leading edge 405 is shown at the top of the trephine. The leading edge 405 extends around the perimeter of the trephine. The leading edge is configured to cut into a donor cornea or cut through a donor cornea. The leading edge is sharp and provides a cutting blade. The cross-sectional shape of the trephine is keyhole shaped. The walls of the tubular trephine form the keyhole shape in cross section.

The cross sectional shape of the trephine is now described with reference to FIG. 6. FIG. 6 shows a view along a longitudinal axis of the trephine. The cross-sectional shape of the trephine is generally keyhole shaped. The walls of the tubular trephine form the perimeter of the keyhole shape. The cross sectional shape of the trephine includes a rounded portion 610. The cross sectional shape of the trephine also includes a tail portion 620 extending outwardly from the rounded portion 610. The tail portion 620 includes a tip 630. The perimeter of the trephine forms a keyhole shape in cross section. The cross section of the trephine forms a keyhole shape having a rounded portion and an elongated tail portion extending from the rounded portion.

The cross-section of the trephine has a major axis 640 extending between the tip 630 of the tail portion on the perimeter of the trephine and an oppositely opposed surface 635 of the rounded portion on the perimeter of the trephine. The major axis of the trephine encompasses the rounded portion and the tail portion. The cross-section of the trephine is generally symmetrical about the major axis 640. The shape of the cross-section is mirrored about the major axis 640.

The cross-section of the trephine includes a minor axis 645 set perpendicularly to the major axis 640. Minor axis 645 extends across the rounded portion 610 of the trephine between opposing surfaces of the perimeter of the trephine. In an embodiment in which the rounded portion 610 is circular, the minor axis 645 forms a diameter of the rounded portion. The cross-section of the trephine is generally asymmetrical about the minor axis. Generally, one side of the minor axis includes the rounded portion only (for example the section shown below the minor axis in FIG. 6) and the other side of the minor axis includes the tail portion and part of the rounded portion (for example the section shown above the minor axis in FIG. 6).

The major axis of the trephine has a greater dimension than the minor axis of the trephine.

The rounded portion of the trephine may be circular, oval or formed from another shape.

The tail portion extends from the rounded portion 610 and may be shaped in different configurations. The tail portion has a base and a tip. The tail portion 620 extends between a base, at the intersection between the tail portion and the rounded portion, and a tip 630 defined on the perimeter of the trephine at the point of the tail where the major axis intersects the perimeter of the trephine.

The tail has a width 625 extending across the perimeter of the trephine at an angle perpendicular to the major axis 640. In some embodiments the width of the tail is greater at the base of the tail compared with at the tip of the tail. This may be a tapered configuration from the base of the tail towards the tip. The taper may be constant along the tail or the taper may vary along the tail. The greater width at the base can provide greater strength in a corneal graft cut using the trephine. The configuration provides a more robust structure and can help prevent the tail from separating from the rounded portion during preparation of the graft. This configuration can help avoid weakness at the base which could result in the tail tearing off during or after the graft preparation process. In FIG. 6, the width of the tail at the base 627 is greater than the width at the tip 628. The tail may have a constant width from the base to the tip. The corners of the tip of the tail 631 632 may be curved.

The perimeter of the keyhole shape at the edge of the trephine defines the leading edge of the trephine. The perimeter is a continuous perimeter. The leading edge is sharpened to form a cutting blade. The cutting blade defines the leading edge of the trephine.

The trephine has a wall thickness 660 defined between an inner surface 661 and an outer surface 665. The cutting blade is positioned on the inside surface of the trephine. The leading edge of the trephine is sharpened from the outer surface forming a taper inwardly from the outer surface towards the inner surface. The taper forms a cutting edge along the interface of the taper and the inner surface. The cutting edge is positioned around the perimeter of the trephine on the inner surface. In other embodiments the cutting edge may be formed around the perimeter of the trephine on the outer surface. In other embodiments the cutting edge may be formed between the inner surface and the outer surface. A taper may extend from the inner surface to the cutting edge and from the outer surface to the cutting edge. The cutting edge may be at the interface of the two tapers.

Referring now to the side view of the trephine shown in FIG. 4. The side view of FIG. 4 shows the tail portion of the trephine 422 and the rounded portion 420. The side view shows the trephine extending between the tip of the tail portion 412 and the point of the perimeter 410 corresponding to an oppositely opposed surface 640 of the rounded portion on the perimeter of the trephine. The leading edge of the trephine has a convex curvature. The curvature is configured to match the curvature of the cornea. The convex curvature is symmetric about the major axis.

The leading edge has a convex curvature that extends from low points at, or proximate to, the points on the perimeter of the leading edge at which the major axis intersects the perimeter of the trephine to a crest at a point on the perimeter at or towards the mid point of the major axis. The convex curvature is best seen in the side view of FIG. 4. The convex curvature is also shown in FIG. 5.

The convex curvature of the leading edge extends from low points 410 412 to crest points 414 414′ between the low points on the perimeter of the leading edge. The low points are positioned at or about the points on the perimeter of the leading edge where the major axis intersects the perimeter. The curvature extends from the low points to create crest points positioned on the perimeter between the low points. The curvature is symmetrical about the major axis.

The low points are at a common height relative to the crest. The crest is positioned at or around the midpoint of the major axis. The position of the crest with respect to the low points is best shown in FIGS. 4 and 5. In embodiments the crest is positioned at the midpoint of the major axis. The curvature is symmetrical about the major axis.

The trephine is configured to cut a corneal graft from a cornea of a donor eye. The cornea is cut after removal of the eye. Typically the cornea is cut from the eye and positioned to sit in a punch block. The cornea has a natural curvature. The punch block is bowl shaped and is configured to receive the cornea and to match the curvature of the cornea. This allows the cornea to sit within the punch block. The outer surface of the cornea sits in contact with the surface of the punch block. Preferably the punch block is configured so the outer surface of the cornea sits flush with the surface of the punch block. The inner surface of the cornea includes delicate endothelial cells. The endothelial cells are on the inside of the cornea and so do not come into contact with the punch block.

The total thickness of the cornea is typically 600 microns to 700 microns. The thickness of the endothelial cells on the inner surface of the cornea are typically 50 microns.

In order to punch a corneal graft from the cornea, the cutting edge of the trephine is moved towards the inner surface of the cornea. The cutting edge of the trephine is brought into contact with the inner surface of the cornea and pressed towards the punch block. The cutting edge of the trephine cuts through the cornea and may be brought into contact with the punch block.

The trephine may include an alignment device to maintain alignment of the trephine with the punch block. The alignment device may be configured to engage with a corresponding alignment means on the punch block to maintain alignment between the punch block and the trephine.

The curvature of the cutting blade is configured to match the curvature of the cornea. The curvature of the cutting blade is configured to match the curvature of the punch block. When the cutting blade is brought into contact with the inside surface of the cornea, all parts of the perimeter contact the inner surface of the cornea simultaneously. The perimeter of the tail portion and the rounded portion of the trephine punch the cornea at the same time. Simultaneous contact of the cutting edge will the cornea at all parts of the perimeter can prevent tearing of the cornea graft.

By positioning the crest of the cutting blade on the perimeter at or towards the mid point of the major axis, the entire trephine can fall inside the cornea of 11-12 mm diameter. This enables the full graft (disc portion and tail portion) to be endothelial cells. If the crest of the cutting blade was aligned with the centre of the rounded portion of the trephine, then the tail would include scleral. This decentration was achieved by changing the curve so that the lowest point was close to where the tail was starting.

The trephine is formed from surgical grade material. Examples of suitable materials include steel, including stainless steel and titanium alloys.

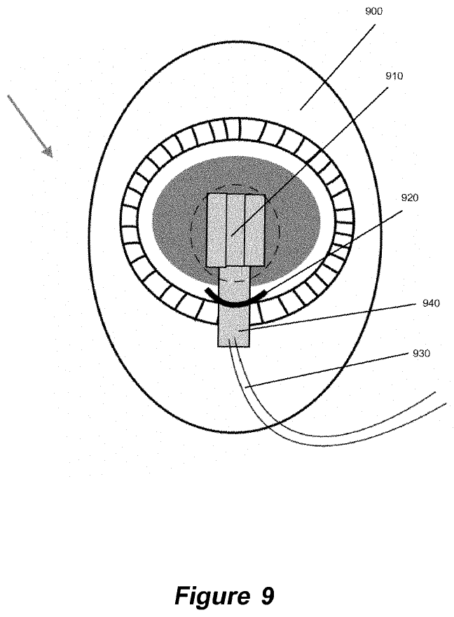

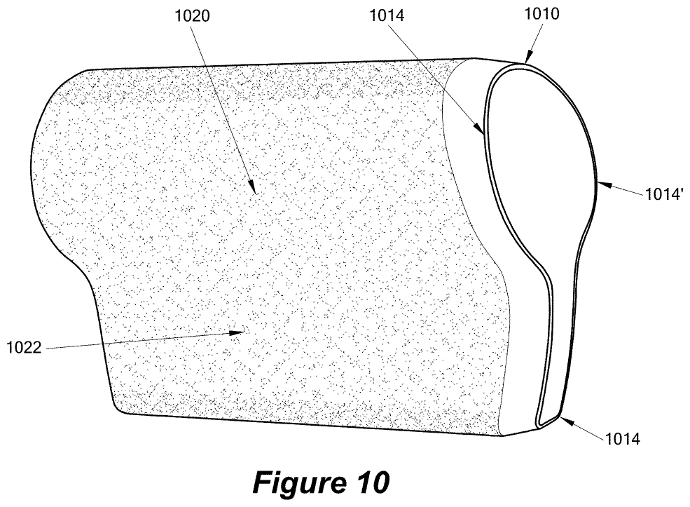

The method for conducting the corneal transplant is now discussed with reference to FIG. 7, FIG. 8 and FIG. 9. The corneal implant tends to roll up into a scroll configuration after the endothelia cells have been removed from the stroma due to the very thin nature of the tissue (5 to 15 microns) as shown in FIG. 7. Preferably the figure is arranged such that the endothelial cells are on the outside of the scrolled implant naturally.

In preferred embodiments an ink stain is added to the implant, for example on the tail, on either the top or bottom surface to enable the surgeon to identify orientation of the implant and identify which surface includes the endothelial cells.

In further embodiments, convex or concave indentations are used as orientation marks on the graft. In further embodiments additional tails can be used to orient the graft prior to injection. The use of convex or concave indentations may be used as an alternative to ink stains or in addition to ink stains.

The use of indentations is a permanent solution which would be non-toxic. Convex indentations may be stronger and prevent the graft from tearing while concave indentations would not catch on any edges.

Further embodiments use further orientation features.

In event the tail breaks then the orientation marks can still be used to orient the graft and continue the surgery using conventional techniques.

In order to conduct the surgery the patient's cornea is prepared by making an incision 800 in the patient's eye. The incision is made in the cornea. The posterior region of the cornea is prepared by removing the damaged endothelial cells in the region required for endothelial transplant. This procedure is called a descementorhexis. The area of the cornea from which the diseased Descemet endothelial complex has to be removed is marked on the cornea. Multiple small incisions are made less than 1 mm in width are made at the cornea scleral limbus. The anterior chamber can be filled with air which is the preferred method or balanced salt solution. A reverse Synsky hook is inserted through the small incisions which prevents air or fluid escape and the edge of the endothelial disc to be removed is marked. The disc is scraped and mobilized toward the main incision (80-0). The endothelial cells are removed from the eye using a blade inserted through incision 800.

Cornea implant is inserted through incision 800 into the anterior chamber with the aim of occupying the denuded space. The corneal implant is centered and unrolled with the endothelial tissue of the corneal implant facing the posterior surface of the cornea in order to adhere to the patient's cornea.

The embodiment of FIG. 8 shows corneal implant 810 loaded within delivery device 820 which may be in the form of a syringe or cartridge. The syringe or cartridge also includes fluid around the corneal implant to prevent it being damaged by the delivery device. In the example of FIG. 8 a suture is placed in the tail of the corneal implant in order to be able to accurately control the movement of the corneal implant within the syringe and from the syringe into the interior chamber of the patient's eye 830.

In the embodiment of FIG. 8 a further incision is made in the cornea at the opposite side of the eye from the first incision in order to manage the flow of fluid from the eye. In a further embodiment the incisions used for the descemetorhexis can be used for the same purpose. This management of fluid through the eye assists in controlling the positioning of the corneal implant. The delivery system is connected to an irrigation line of a phaco emulsification machine. The system is set to a slow aspiration rate and low bottle height i.e. continuous irrigation. The corneal implant is delivered slowly releasing the suture. The corneal implant is arranged such that the tail portion remains behind within the syringe. In an alternative embodiment the corneal implant is delivered using the fluid in a syringe without continuous irrigation described above.

The tail portion of the corneal implant is used as the manipulating portion to which the suture may be attached. A benefit of using the tail portion as the manipulating region is that, since no contact is required to be made to the endothelial cells in the disc region, these cells are not damaged. Additionally, the sclera portion of the corneal implant tends to have greater integrity and is easier to manipulate than the endothelial portion.

The corneal implant is delivered into the interior chamber and the disc portion is centered into position for adhering to the posterior surface of the cornea. FIG. 9 shows a top view of an eye after the corneal implant 910 has been inserted into the interior chamber of the eye through incision 920. As illustrated in FIG. 9 after insertion corneal implant remains in the scrolled configuration. FIG. 9 also shows suture attached to tail 940 of the corneal implant. The incision 930 could also be closed with a suture to prevent accidental egress of the corneal implant out of the graft.

By manipulating the corneal implant in this controlled manner the orientation can be ensured by keeping the tail orientated correctly. The tail can be manipulated to center the corneal implant. If the tail inadvertently enters the eye, the suture may be used to draw the tail out. Unrolling the scrolled corneal implant may be achieved by moving an air bubble above the corneal implant although this may not be necessary when the width of the tail is significant in size compared with the disc portion. The use of air and tapping on the cornea is a well-established technique for unrolling the eye. Air is then injected deep into the corneal implant to aplanate it to the posterior surface of the recipient cornea. The air would be left in-situ for 30 mins. After the corneal implant has been inserted, positioned and unrolled, a stitch is inserted into the incision in order to seal the interior chamber of the eye. The tail portion of the corneal implant protrudes from the incision (see 940) and so can also be stitched and held in place. The stitching also retains the position of the corneal implant in order to aid with adhesion to the posterior surface of the cornea. In another embodiment of the procedure a stitch may be preplaced prior to injection of the graft so as not to disturb the wound once the graft is in place. This suture can be tightened after graft insertion and unrolling.

The protruding section of the tail may be cut or torn along the perforated section. In another embodiment a small needle can be inserted into the anterior chamber after graft adhesion and perforations can be created by the needle and the tail can then be removed. In another embodiment this could be performed post-operatively in the clinic after the graft adhesion is ensured. If the graft does not include perforations the tail can be cut or removed in another way.

In another embodiment the graft is loaded in an injector such that the tail emerges first. A forcep is introduced from the opposite side of the cornea through an incision into the anterior chamber to grasp and drag the graft by the tail. The tail is externalised delivering the graft into the anterior chamber with the tail outside. The incision is sutured. Various known techniques are used to unroll the graft. The tail is moved to manipulate the graft into unrolling and for centring the graft. The markings on the graft are used to reconfirm correct orientation. Once the graft is unrolled, air is injected deep to the graft to applanate the graft to the cornea.

It will be clear to those skilled in the art that embodiments of the present invention provide a corneal implant and method of surgery which enables a corneal implant to be inserted and manipulated within the anterior chamber of the eye and positioned to enable the transplant of endothelial tissue. The inclusion of a tail portion within the corneal implant provides a region of the implant which can be manipulated without fear of damaging important endothelial cells required for transplant. This helps increase the probability of conducting a successful surgery.

The specific embodiments described above are not limiting to the scope of the invention. In particular, the shape of the corneal implant is not limited to a keyhole configuration having a disc of endothelial tissue and a rectangular shaped tail including at least a portion of sclera. Instead, alternative shapes could be used which include a transplant portion including endothelial cells and a manipulating portion. The disc may be a semi-circle or cresenteric or annulus or star shaped to prevent the graft from rolling or to apply the graft to only those areas that need the tissue. The tail may be rectangular or triangular with a narrow portion near the disc or away from the disc. Alternatively any suitable shaped implant may be used.

Further embodiments of the corneal implant may have more than one tail or other notches or convex protrusions for manipulation, assistance with unrolling and for orientation.

Other shapes of the corneal implant may be used.

Embodiments can be used for other types of corneal transplants such as anterior lamellar keratoplasty with multiple tails to help anchor the graft and for grafts elsewhere in the body.

The shape of the transplant may be achieved by cutting with a blade or laser or printed on a scaffold to achieve a configuration mentioned above.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art, in Australia or any other country.

What is claimed is:

1. A corneal trephine comprising a tubular body and a cross section having a rounded portion and an elongated tail portion extending from the rounded portion, the tubular body having a leading edge configured to cut into a cornea.

2. A corneal trephine according to claim 1, the cross section having a major axis and a minor axis, the major axis encompassing the tail portion and the rounded portion.

3. A corneal trephine according to claim 2, the cross section being symmetrical about the major axis.

4. A corneal trephine according to claim 1 the leading edge having a convex curvature.

5. A corneal trephine according to claim 4 wherein the convex curvature is configured to match the curvature of a cornea.

6. A corneal trephine according to claim 4 wherein the convex curvature of the leading edge extends from low points on the leading edge at or around the points at which the major axis intersects the leading edge to crest points on the leading edge at or towards a mid point of the major axis.

7. A corneal trephine according to claim 4 wherein the complex curvature is symmetrical about the major axis.

8. A corneal trephine wherein the leading edge is a cutting blade.

9. A kit comprising a corneal trephine according to claim 1 and a punch block, the punch block having a curved surface, wherein the curvature of the leading edge is complimentary to the curved surface of the punch block.

10. A kit according to claim 9 further comprising an alignment device to align the trephine with respect to the punch block.
